(19) United States
(12) Patent Application Publication
(10) Pub. No.: US 2026/0086078 A1
(43) Pub. Date: Mar. 26, 2026

(54) METHOD FOR DETERMINATION OF WHOLE WASTEWATE TOXICITY

(71) Applicant: Nanjing University, Nanjing (CN)

(72) Inventors: Haidong HU, Nanjing (CN); Aixia ZHAO, Nanjing (CN); Kewei LIAO, Nanjing (CN); Hongqiang REN, Nanjing (CN)

(21) Appl. No.: 18/936,972

(22) Filed: Nov. 4, 2024

(30) Foreign Application Priority Data
Sep. 20, 2024 (CN) 202411320177.X

(51) Int. Cl. G01N 33/18; G01N 33/50

(52) U.S. Cl. CPC G01N 33/1826; G01N 33/5014; G01N 2333/4603

(57) ABSTRACT

A method for determination of whole wastewater toxicity including: measuring the zebrafish embryo toxicity indexes of wastewater samples full-scale of a plurality of wastewater treatment plants and preprocessing obtained data, the zebrafish embryo toxicity indexes including traditional toxicity indexes and behavioral toxicity indexes; establishing whole wastewater toxicity prediction models for wastewater based on different target variables, with the traditional toxicity indexes as target variables and the behavioral toxicity indexes as features; and inputting behavioral toxicity index data of zebrafish embryos of a to-be-tested wastewater sample into the whole wastewater toxicity prediction models; selecting a corresponding prediction model based on a prediction result of the target variables, to obtain a whole wastewater toxicity of the to-be-tested wastewater sample.

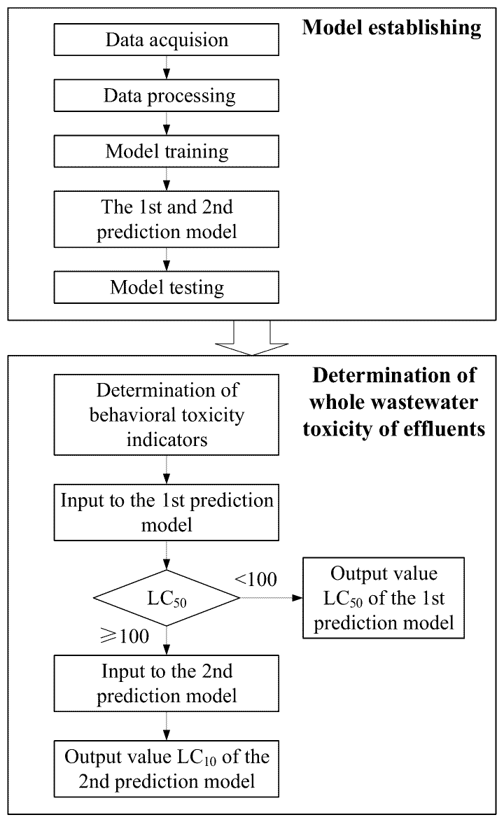

### CROSS-REFERENCE TO RELATED APPLICATIONS

Pursuant to 35 U.S.C. § 119 and the Paris Convention Treaty, this application claims foreign priority to Chinese Patent Application No. 202411320177.X filed Sep. 20, 2024, the contents of which, including any intervening amendments thereto, are incorporated herein by reference. Inquiries from the public to applicants or assignees concerning this document or the related applications should be directed to: Matthias Scholl P.C., Attn.: Dr. Matthias Scholl Esq., 245 First Street, 18th Floor, Cambridge, MA 02142.

### BACKGROUND

The disclosure relates to the field of water quality monitoring, and more particularly to a method for determination of whole wastewater toxicity.

Human activities result in the dispersion of many pollutants into the environment, which may pose potential risks to human health and the environment. Traditional chemical methods are often used to measure specific pollutants in wastewater. However, wastewater often exists as complex mixtures, many constituents cannot be identified by chemical analysis, thus increasing uncertainty in water quality assessment. In contrast to chemical analysis, bioanalysis is able to measure the actual toxicity of all pollutants in an integrated manner, i.e., whole wastewater toxicity.

Among the bioanalytical methods, zebrafish embryos are widely used due to their rapid reproductive cycle and transparent nature. The whole wastewater toxicity index obtained by the traditional experimental method is LC₁₀ or LC₅₀ (unit: percentage of water sample concentration), which is standardized and widely accepted. However, this method involves multiple concentration gradients and multiple parallel experiments, which increases the amount of embryos used and the experimental operation time. To solve the problem, a method for assessing developmental neurotoxicity of zebrafish embryos in wastewater is developed. The method exposes transgenic zebrafish embryos to concentrated or diluted wastewater to be tested up to 24 hours post-fertilization (hpf), and then the exposed embryos are collected and observed under a fluorescence inverted microscope for image analysis, which has the advantages of simple operation, rapidity and efficiency. However, the water samples need to be filtered and concentrated, which may not reflect the real wastewater; and the method can only obtain the neurotoxicity index, but not the whole wastewater toxicity.

### SUMMARY

One objective of the disclosure is to provide a method for the rapid determination of whole wastewater toxicity, which is capable of obtaining standardized indexes of whole wastewater toxicity while maintaining the advantages of speed, simplicity and low cost, so that the method is applicable to the rapid determination of whole wastewater toxicity of large quantities of actual wastewater samples.

The disclosure provides a method for determination of whole wastewater toxicity, the method comprising:
1) measuring the zebrafish embryo toxicity indexes of wastewater samples full-scale of a plurality of wastewater treatment plants and preprocessing obtained data, the zebrafish embryo toxicity indexes comprising traditional toxicity indexes and behavioral toxicity indexes;
2) establishing whole wastewater toxicity prediction models for wastewater based on different target variables, with the traditional toxicity indexes as target variables and the behavioral toxicity indexes as features; and
3) inputting behavioral toxicity index data of zebrafish embryos of a to-be-tested wastewater sample into the whole wastewater toxicity prediction models; selecting a corresponding prediction model based on a prediction result of the target variables, to obtain a whole wastewater toxicity of the to-be-tested wastewater sample.

In a class of this embodiment, the traditional toxicity indexes comprise a medium lethal concentration (LC₅₀) and a 10% lethal concentration (LC₁₀), which are measured by observing a mortality rate of zebrafish embryos within 48 hpf.

In a class of this embodiment, in 2), establishing whole wastewater toxicity prediction models for wastewater based on different target variables comprises: according to different target variables, selecting wastewater samples with LC₅₀<100 and training with LC₅₀ as a target variable to yield a first prediction model; selecting wastewater samples with LC₅₀≥100 and training with LC₁₀ as a target variable to yield a second prediction model.

In a class of this embodiment, in 3), selecting a corresponding prediction model based on a prediction result of the target variables, to obtain a whole wastewater toxicity of the to-be-tested wastewater sample comprises:
inputting the behavioral toxicity index data of zebrafish embryos of the to-be-tested wastewater sample into the first prediction model, if an output value LC₅₀ of the first prediction model is <100, then the LC₅₀ value is the whole wastewater toxicity of the to-be-tested wastewater sample; if the output value LC₅₀ of the first prediction model is ≥100, inputting the behavioral toxicity index data of zebrafish embryos of the to-be-tested wastewater sample into the second prediction model, and an output value LC₁₀ of the second prediction model is the whole wastewater toxicity of the to-be-tested wastewater sample.

In a class of this embodiment, the behavioral toxicity indexes comprise zebrafish embryo activity under dark conditions within 120 hpf, zebrafish embryo activity under bright conditions within 120 hpf, total movement distance of zebrafish embryo under dark conditions within 120 hpf, total movement distance of zebrafish embryo under bright conditions within 120 hpf, zebrafish embryo bursting distance within 120 hpf, zebrafish embryo cruising distance within 120 hpf, and zebrafish embryo freezing distance within 120 hpf, which are measured using a zebrafish behavioral detection instrument.

In a class of this embodiment, the whole wastewater toxicity prediction models for wastewater are established with Lasso model.

In a class of this embodiment, during establishing the whole wastewater toxicity prediction models, the performance of the whole wastewater toxicity prediction models is evaluated through a determination coefficient R², which is calculated using the following method:

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - {\overline{y}}_{i} )}^{2}} ;$$

R² is the coefficient of determination, yᵢ is an i-th traditional toxicity index value in a test set, y̅ is an average value of the traditional toxicity index in the test set, y̅ᵢ is a predicted value output by the prediction model, and n is a number of traditional toxicity index values.

In a class of this embodiment, preprocessing obtained data comprises data cleaning, data normalization, and feature selection.

In a class of this embodiment, the data normalization employs Z-Score method or Max-Min method.

In a class of this embodiment, the feature selection employs Pearson correlation coefficient method.

The following advantages are associated with the method for determination of whole wastewater toxicity of the disclosure.
1. The method of the disclosure can quickly and simply realize the toxicity testing of large quantities of wastewater samples, and the number of embryos used is greatly reduced compared with traditional experiments, thus reducing the cost.
2. The prediction model is selected according to the target variable prediction results, which improves the accuracy of the model in the practical application.
3. The determination coefficient R² of established model of the disclosure is >0.85, with high accuracy.

### BRIEF DESCRIPTION OF THE DRAWINGS

The sole FIGURE is a flow chart of a method for determination of whole wastewater toxicity in accordance with one embodiment of the disclosure.

### DETAILED DESCRIPTION

To further illustrate the disclosure, embodiments detailing a method for determination of whole wastewater toxicity are described below. It should be noted that the following embodiments are intended to describe and not to limit the disclosure.

As shown in the sole FIGURE, the disclosure provides a method for determination of whole wastewater toxicity, the method comprising establishing whole wastewater toxicity prediction models for wastewater (1) and measuring whole wastewater toxicity (2).

Specifically, establishing whole wastewater toxicity prediction models for wastewater (1) is achieved as follows:

### (1.1) Measuring the Zebrafish Embryo Toxicity Indexes of Wastewater Samples Full-Scale of a Plurality of Wastewater Treatment Plants, the Zebrafish Embryo Toxicity Indexes Comprising Traditional Toxicity Indexes and Behavioral Toxicity Indexes

Optionally, the traditional toxicity indexes comprise a medium lethal concentration (LC₅₀) and a 10% lethal concentration (LC₁₀), which are measured by observing a mortality rate of zebrafish embryos within 48 hpf.

The behavioral toxicity indexes comprise zebrafish embryo activity under dark conditions (active-dark) within 120 hpf, zebrafish embryo activity under bright conditions (active-bright) within 120 hpf, total movement distance of zebrafish embryo under dark conditions (total distance-dark) within 120 hpf, total movement distance of zebrafish embryo under bright conditions (total distance-bright) within 120 hpf, zebrafish embryo bursting distance within 120 hpf, zebrafish embryo cruising distance within 120 hpf, and zebrafish embryo freezing distance within 120 hpf, which are measured using a zebrafish behavioral detection instrument.

(1.2) Preprocessing Obtained Data, that is, Data Cleaning, Data Normalization, and Feature Selection.

The data normalization includes but is not limited to Z-Score method or Max-Min method.

In this embodiment, the Z-Score method is used, and the calculation method is as follows:

$$Z = \frac{( x - μ )}{σ} ;$$

where Z is a standard score, x is an indicator value in the behavioral toxicity index, μ is a mean of the data of the indicator value, and σ is a standard deviation of the indicator value.

The feature selection method uses the Pearson correlation coefficient determination method, and if the correlation coefficient r² of the two indicators is >0.8, then any one of the indicators is deleted, so that the correlation coefficient r² between every two features is <0.8.

The processed data is divided into a training set and a test set.

### (1.3) Model Establishing

Taking traditional toxicity indicators as target variables and behavioral toxicity indicators as features, the whole wastewater toxicity prediction model for wastewater was established based on the Lasso model.

### (1.4) Model Training and Testing

The prediction model is trained using the training set data; and the prediction model performance is evaluated based on the determination coefficient R² using the test set.

The determination coefficient R² is calculated using the following method:

$${R}^{2} = 1 - \frac{{\sum }_{i = 1}^{n} {( {y}_{i} - {\hat{y}}_{i} )}^{2}}{{\sum }_{i = 1}^{n} {( {y}_{i} - {\overline{y}}_{i} )}^{2}} ;$$

R² is the coefficient of determination, yᵢ is an i-th traditional toxicity index value in a test set, y̅ is an average value of the traditional toxicity index in the test set, ŷᵢ is a predicted value output by the prediction model, and n is a number of traditional toxicity index values.

Specifically, depending on the target variable, the wastewater samples with LC₅₀<100 (i.e., the concentration of water samples is 100%) are selected and trained with LC₅₀ as the target variable to obtain a first prediction model; the wastewater samples with LC₅₀≥100 are selected and trained with LC₁₀ as the target variable to obtain a second prediction model.

Measuring whole wastewater toxicity (2) is achieved as follows:
(2.1) Determination of behavioral toxicity indexes of zebrafish embryos of water samples to be tested: zebrafish embryos are utilized to determine the behavioral toxicity indexes of wastewater samples to be tested; and the obtained data are pre-processed;
(2.2) The pre-processed behavioral toxicity index data are input into the first prediction model, if the output value of the first prediction model LC₅₀<100, then the value of LC₅₀ is the whole wastewater toxicity of the water sample;
if the output value LC₅₀ of the first prediction model is ≥100, the embryonic behavioral toxicity index data of the water sample to be tested is input into the second prediction model, and the output value LC₁₀ of the second prediction model is obtained, which is the whole wastewater toxicity of the water sample.

The method of the disclosure is further illustrated through the following example.

The effluent samples used to establish the whole wastewater toxicity prediction models were from a plurality of process sections of 40 wastewater plants, and the number of the effluent samples was 300. The wastewater samples to be tested were actual influent and actual effluent from a municipal wastewater plant, and the method for rapid determination of whole wastewater toxicity of the wastewater of the disclosure comprises the following steps.
(1) Establishment of prediction models for LC₅₀ and LC₁₀ based on behavioral data of zebrafish embryos
(1.1) Data collection: Zebrafish embryo toxicity of 300 effluent samples were measured, including traditional toxicity indicators and behavioral toxicity indicators. Traditional toxicity indicators included LC₅₀ and LC₁₀, measured by observing embryonic mortality at a specific time; in this example, there were 130 effluent samples with LC₅₀<100 and 170 effluent samples with LC₅₀≥100. The behavioral toxicity indicators included embryonic activity under 5-minute dark conditions, embryonic activity under 5-minute light conditions, total movement distance under 5-minute dark conditions, total movement distance under 5-minute light conditions, and bursting distance, cruising distance, and freezing distance under 5-minute dark and 5-minute light conditions, which were measured by using a zebrafish behavioral tester.
(1.2) Data pre-processing: data cleaning, data standardization and feature selection were performed on the collected data. Data standardization adopted Z-Score value method; feature selection method adopted Pearson correlation coefficient determination, and the final correlation coefficient r² between every two features ranges from 0.496 to 0.770.
(1.3) Model training: the dataset was divided into 70% training set and 30% test set, with traditional toxicity indicators as target variables and behavioral toxicity indicators as features, and trained with the Lasso mode. There were 130 water samples with LC₅₀<100 and 170 water samples with LC₅₀≥100, so there were 130 water samples trained with LC₅₀ as target variable to get the first prediction model, and 170 water samples were trained with LC₁₀ as the target variable to obtain the second prediction model.
(1.4) Model Testing: Evaluate the model performance based on the determination coefficient R² using the test set data according to the trained model. The first prediction model: R²=0.874. The second prediction model: R²=0.893.
(2) Determination of whole wastewater toxicity of effluents using established models.
(a) When the effluent sample to be tested was an actual influent of a municipal wastewater plant:
(2.1a) Determination of behavioral toxicity indicators: zebrafish embryos were used to determine the behavioral toxicity indicators of the effluent samples to be tested, and 10 parallel groups were set up for each water sample, and the data on behavioral toxicity indicators obtained are shown in Table 1.

TABLE 1
Behavioral toxicity indicators of the effluent samples to be tested

| | | | Total | Total | | | |
|---|---|---|---|---|---|---|---|
| | Active- | Active- | distance- | distance- | Bursting | Cruising | Freezing |
| | bright | dark | bright | dark | distance | distance | distance |
| | (mm · s) | (mm · s) | (mm) | (mm) | (mm) | (mm) | (mm) |
| 1 | 4.0 | 65.0 | 235.0 | 130.9 | 244.1 | 118.8 | 3.1 |
| 2 | 7.0 | 1795.0 | 148.0 | 514.1 | 413.2 | 117.3 | 131.5 |
| 3 | 7.0 | 593.0 | 117.4 | 206.3 | 280.0 | 14.5 | 29.3 |
| 4 | 9.0 | 520.0 | 63.2 | 202.0 | 194.8 | 44.3 | 26.0 |
| 5 | 11.0 | 1697.0 | 148.8 | 436.4 | 427.7 | 103.9 | 53.7 |
| 6 | 14.0 | 337.0 | 66.6 | 160.2 | 120.5 | 45.2 | 61.2 |
| 7 | 15.0 | 27.0 | 67.3 | 125.0 | 181.1 | 9.6 | 1.7 |
| 8 | 15.0 | 338.0 | 105.4 | 103.0 | 175.1 | 13.5 | 19.7 |
| 9 | 17.0 | 462.0 | 54.4 | 193.5 | 95.9 | 36.7 | 115.3 |
| 10 | 27.0 | 108.0 | 115.8 | 103.0 | 110.5 | 93.6 | 14.8 |
| Average | 12.6 | 594.2 | 112.2 | 217.4 | 224.3 | 59.7 | 45.6 |

(2.2a) Model selection: the average of the obtained behavioral toxicity indicators was input data into the first prediction model to obtain LC₅₀=65 for the influent water, and since LC₅₀<100, LC₅₀=65 was the whole wastewater toxicity of the water sample.
(b) When the effluent sample to be tested was an actual effluent from a municipal wastewater plant:
(2.1b) Determination of behavioral toxicity indicators: zebrafish embryos were used to determine the behavioral toxicity indicators of the wastewater samples to be tested, each water sample was set up in 10 parallel groups, and the behavioral toxicity indicators data obtained are shown in Table 2.

TABLE 2
Behavioral toxicity indicators of the effluent samples to be tested

| | | | Total | Total | | | |
|---|---|---|---|---|---|---|---|
| | Active- | Active- | distance- | distance- | Bursting | Cruising | Freezing |
| | bright | dark | bright | dark | distance | distance | distance |
| | (mm · s) | (mm · s) | (mm) | (mm) | (mm) | (mm) | (mm) |
| 1 | 151.0 | 2276.0 | 63.0 | 349.3 | 266.4 | 47.5 | 98.3 |
| 2 | 152.0 | 4638.0 | 42.6 | 520.6 | 287.4 | 101.7 | 174.2 |
| 3 | 158.0 | 1256.0 | 99.8 | 230.0 | 234.7 | 27.7 | 67.3 |
| 4 | 167.0 | 3535.0 | 145.8 | 558.7 | 358.0 | 154.3 | 192.1 |
| 5 | 180.0 | 1598.0 | 179.2 | 527.8 | 447.5 | 140.1 | 119.4 |
| 6 | 183.0 | 3963.0 | 113.6 | 499.5 | 366.0 | 112.7 | 134.4 |
| 7 | 190.0 | 9905.0 | 133.5 | 777.1 | 374.6 | 195.4 | 340.7 |
| 8 | 120.0 | 5070.0 | 93.2 | 673.2 | 236.5 | 164.4 | 365.5 |
| 9 | 132.0 | 14211.0 | 165.0 | 1172.4 | 421.9 | 220.2 | 695.3 |
| 10 | 148.0 | 5064.0 | 194.5 | 849.1 | 420.3 | 199.2 | 424.1 |
| Average | 158.1 | 5151.6 | 123.0 | 615.8 | 341.3 | 136.3 | 261.1 |

(2.2b) Model selection: the average of the obtained behavioral toxicity index was input into the first prediction model to obtain the LC₅₀=136 of the effluent water. Since the LC₅₀ of the effluent water >100, it means that the toxicity of the water sample to be tested was low and it was not suitable to use the LC₅₀ as a toxicity index, therefore, the parameter of the behavioral toxicity index of the effluent water was input into the second prediction model, and LC₁₀=43 was obtained, which was the whole wastewater toxicity index of the water sample.

It will be obvious to those skilled in the art that changes and modifications may be made, and therefore, the aim in the appended claims is to cover all such changes and modifications.

What is claimed is:

1. A method for determination of whole wastewater toxicity, the method comprising:
1) measuring zebrafish embryo toxicity indexes of wastewater samples full-scale of a plurality of wastewater treatment plants and preprocessing obtained data, the zebrafish embryo toxicity indexes comprising traditional toxicity indexes and behavioral toxicity indexes;
2) establishing whole wastewater toxicity prediction models for wastewater based on different target variables, with the traditional toxicity indexes as target variables and the behavioral toxicity indexes as features; and
3) inputting behavioral toxicity index data of zebrafish embryos of a to-be-tested wastewater sample into the whole wastewater toxicity prediction models; selecting a corresponding prediction model based on a prediction result of the target variables, to obtain a whole wastewater toxicity of the to-be-tested wastewater sample.

2. The method of claim 1, wherein the traditional toxicity indexes comprise a medium lethal concentration (LC₅₀) and a 10% lethal concentration (LC₁₀), which are measured by observing a mortality rate of zebrafish embryos within 48 hours post-fertilization (hpf).

3. The method of claim 2, wherein in 2), establishing whole wastewater toxicity prediction models for wastewater based on different target variables comprises: according to different target variables, selecting wastewater samples with LC₅₀<100 and training with LC₅₀ as a target variable to yield a first prediction model; selecting wastewater samples with LC₅₀≥100 and training with LC₁₀ as a target variable to yield a second prediction model.

4. The method of claim 3, wherein in 3), selecting a corresponding prediction model based on a prediction result of the target variables, to obtain a whole wastewater toxicity of the to-be-tested wastewater sample comprises:
inputting the behavioral toxicity index data of zebrafish embryos of the to-be-tested wastewater sample into the first prediction model, if an output value LC₅₀ of the first prediction model is <100, then the LC₅₀ value is the whole wastewater toxicity of the to-be-tested wastewater sample; if the output value LC₅₀ of the first prediction model is ≥100, inputting the behavioral toxicity index data of zebrafish embryos of the to-be-tested wastewater sample into the second prediction model, and an output value LC₁₀ of the second prediction model is the whole wastewater toxicity of the to-be-tested wastewater sample.

5. The method of claim 1, wherein the behavioral toxicity indexes comprise zebrafish embryo activity under dark conditions within 120 hpf, zebrafish embryo activity under bright conditions within 120 hpf, total movement distance of zebrafish embryo under dark conditions within 120 hpf, total movement distance of zebrafish embryo under bright conditions within 120 hpf, zebrafish embryo bursting distance within 120 hpf, zebrafish embryo cruising distance within 120 hpf, and zebrafish embryo freezing distance within 120 hpf, which are measured using a zebrafish behavioral detection instrument.

6. The method of claim 1, wherein the whole wastewater toxicity prediction models for wastewater are established with Lasso model.

7. The method of claim 1, wherein during establishing the whole wastewater toxicity prediction models, the performance of the whole wastewater toxicity prediction models is evaluated through a determination coefficient R², which is calculated using the following method:
$${R}^{2} = 1 - \frac{{\sum }_{i = 1}^{n} {( {y}_{i} - {\hat{y}}_{i} )}^{2}}{{\sum }_{i = 1}^{n} {( {y}_{i} - {\overline{y}}_{i} )}^{2}} ;$$
R² is the coefficient of determination, yᵢ is an i-th traditional toxicity index value in a test set, y̅ is an average value of the traditional toxicity index in the test set, ŷᵢ is a predicted value output by the prediction model, and n is a number of traditional toxicity index values.

8. The method of claim 1, wherein preprocessing obtained data comprises data cleaning, data normalization, and feature selection.

9. The method of claim 8, wherein the data normalization employs Z-Score method or Max-Min method.

10. The method of claim 8, wherein the feature selection employs Pearson correlation coefficient method.
